(19) United States
(12) Patent Application Publication
(10) Pub. No.: US 2011/0125113 A1
(43) Pub. Date: May 26, 2011

(54) TISSUE ENCLOSURE

(76) Inventor: Carmeli Adahan, Jerusalem (IL)

(21) Appl. No.: 12/999,663

(22) PCT Filed: Jun. 23, 2009
(86) PCT No.: PCT/IL09/00618

§ 371 Date: Dec. 17, 2010

(30) Foreign Application Priority Data
Jun. 23, 2008 (GB) 0811457.1

(51) Int. Cl. A61F 13/02; A61F 13/00; A61B 17/00

(52) U.S. Cl. 604/307; 604/304; 606/213

(57) ABSTRACT

Provided is a device for providing a vacuum to a tissue, including a first tissue cover for covering the tissue and having first peripheral portion with a tissue facing contact surface adapted to be sealingly affixed to a first tissue portion surrounding the tissue, the device being adapted for enabling a vacuum to be selectively applied to the tissue in operation of the device; a substantially contiguous layer of flowable sealing material configured for covering a second tissue portion abuttingly adjacent at least a part of the first peripheral portion, where the sealing material is configured for filling gaps between the contact surface and the first tissue portion that may form leaks, by flowing towards the contact surface under suction provided when the vacuum is applied to the damaged tissue in operation of the device, and where the sealing material is uncoupled with respect to the contact surface at least prior to affixing the contact surface to the first tissue portion; and a second tissue cover covering at least a part of the layer. The device may be provided in the form of a kit. A system and method including the device are also provided.

### FIELD OF THE INVENTION

This invention relates to enclosures for covering a tissue, in particular damaged tissue, including for example wounds, burns and the like, in particular to such enclosures regarding which a vacuum may be applied.

### BACKGROUND OF THE INVENTION

Applying negative pressure to a physiological area such as a wound, for example, enhances drainage of fluids or exudate from the wound and promotes tissue growth and wound healing. This method of healing (known as “cupping”) was exercised since the times of ancient Greek physicians until the 19th century. Applying negative pressure to other physiological areas such as burn, for example, may be beneficial even when there is no drainage.

By way of general background, a number of systems and methods have been developed for providing suctioning and/or for treatment of wounds, for example as disclosed in the following publications.

U.S. Pat. No. 7,198,046 discloses a method of treating tissue damage, and a wound treatment apparatus in which a fluid impermeable wound cover is sealed over a wound site. A screen in the form of an open-cell foam screen or a rigid porous screen is placed beneath the wound cover over the wound. A vacuum pump supplies suction within the wound cover over the treatment site.

U.S. Pat. No. 5,645,081 discloses a method of treating tissue damage and apparatus for same, comprising applying a negative pressure to a wound.

WO 2003/057070 discloses a ventilated bandage system is provided for use with a wound. The system includes a bandage positioned adjacent the wound to create a sealed environment around the wound. A vacuum source of the system is in communication with the bandage to create negative pressure between the bandage and the wound. The system may also include a first passageway or vent in communication with the bandage and with the surrounding atmosphere, and a second passageway in communication with the bandage and with the vacuum source.

US 2006/025727 and WO 2006/052745 each discloses a system for treating a wound with suction, comprising a wound cover, a pump having an input port and an output port, the input port providing suction to the wound via the wound cover, and a reservoir coupled to the output port of the pump. The reservoir is adapted to receive effluent from the wound and the pump is capable of maintaining a controlled level of suction at the wound.

US 2004/064132 discloses a medical device that includes an enclosure to be placed over a wound, adhesively connected to the skin around the wound by a flange. Gauze type packing cylinders can be placed in the wound, and suction is provided either by a bulb type pump, or by a pair of regulators. When regulators are used, one alternates the suction that it provides, and the other is continuous, such that the level of suction is periodically varied.

US 2001/029956 discloses a method of treating tissue damage, comprising applying a negative pressure to a wound, and a wound treatment apparatus in which a fluid impermeable wound cover is sealed over a wound site. A screen made from open-cell foam or a rigid porous screen is placed beneath the wound cover over the wound. A vacuum pump supplies suction within the wound cover over the treatment site.

In WO 2006/025848 a medical device is disclosed comprising a suction portion, having both an inlet valve and an outlet valve, for suctioning fluids from the wound, and a sealing portion for sealing the wound by engaging the skin adjacent the wound, the suction portion and sealing portion being integral with one another. A method for draining a wound is also disclosed.

In WO 03/057307, a wound vacuum therapy dressing kit is provided for use with a wound drainage system having a vacuum source. The kit may include a wound dressing member, a sealing film, and a wound measurement device. The wound dressing member may include a wound contacting surface configured to be in contact with and generally conform to a wound surface of a patient. The member may be adapted to be coupled to the vacuum source for communicating suction from the vacuum source to the wound surface. The sealing film of the kit may be provided for placement over the member and may be configured to adhere to a patient's healthy skin surrounding the wound. The wound measurement device may include a transparent top portion and a transparent bottom portion configured for placement adjacent the wound surface. The top portion may include a drawing surface and a grid associated with the drawing surface.

US 2004/054338 discloses a portable closed wound drainage system that uses a pouch shaped dressing which is inserted into a wound. At least a portion of the outer surface of the pouch is porous to allow exudates to enter. Exudates are removed from the pouch by flexible tubing which is secured inside the pouch at one end, and secured at the other end to a portable drain/suction unit. The pouch contains porous material, and may optionally contain beads and fillers which are antibacterial in nature. The tubing can have a single or multi-lumen structure with perforations in the side walls of the end of the tube that is inserted in the pouch to allow body fluids to enter laterally. The portable drain/suction unit is preferably a portable battery powered device. The pouch and the tube are sealed by a flexible sealing material which is applied to the outer surface of the skin around the periphery of the pouch and the tubing as it exits the pouch.

WO 2008/048527 discloses a manually-activated reduced pressure treatment system, including a substantially rigid housing, and an end cap slidingly received by the housing. An inner chamber is disposed between the end cap and housing, and a volume of the inner chamber is variable in amount depending on the position of the end cap within the housing. The end cap is slidingly movable between an uncompressed position at which the volume of the inner chamber is at a maximum value and a compressed position at which the volume of the inner chamber is at a minimum value. A position indicating member is associated with the end cap and housing to indicate the position of the end cap relative to the housing at predetermined positions between the uncompressed position and the compressed position.

### SUMMARY OF THE INVENTION

According to a first aspect of the invention; there is provided a kit for applying a vacuum to a tissue, which may facilitate treatment of the tissue when this is damaged tissue, for example, the kit comprising:

(A) a first tissue cover defining an internal volume at least in cooperation with the tissue in operation of said kit, said first tissue cover comprising a first peripheral portion adapted to be sealingly affixed to a first tissue portion circumscribing the tissue, said first tissue cover adapted for enabling a vacuum to be selectively applied to said internal volume in operation of said kit;

(B) a flowable sealing material configured for covering in a substantially contiguous layer at least a second tissue portion abuttingly adjacent at least part of said first peripheral portion, said sealing material configured for filling gaps between said first peripheral portion and said first tissue portion that may form leaks when a vacuum is applied to said internal volume, wherein said sealing material is uncoupled with respect to said peripheral portion at least prior to affixing said peripheral portion to said first tissue portion; and

(C) a second tissue cover for covering at least a portion of said layer therewith.

The kit may comprise one or more of the following features in any combination:
The substantially contiguous layer may be abuttingly adjacent the full periphery of the first peripheral portion, i.e., the layer fully circumscribes the first peripheral portion.
The sealing material may be further configured for covering in said substantially contiguous layer an external portion of said first tissue cover adjacent said first peripheral portion.
The kit may further comprise an interface including at least one of medication, lotions, gauze, bandages, packing material for application to the damaged tissue.
The first tissue cover may comprise a first drape having a tissue-facing adhesive layer and a removable protective layer carried against said adhesive layer prior to use, and wherein said first peripheral portion is adapted to be affixed to the first tissue portion via said adhesive layer having first removed said protective layer. For example, the protective layer may comprise any one of a paper strip, tape and a film.
The second tissue cover may comprise a second peripheral portion configured for affixing said second peripheral portion to a third tissue portion adjacent said second tissue portion. The second peripheral portion may be partially or fully circumscribing the second tissue portion.
The kit may further comprise an applicator for applying said sealing material to at least said second tissue portion.
The sealing material may comprise for example any one of petroleum jelly, vacuum grease.
The first tissue cover may comprise a conduit having at one end thereof an inlet port opening into said internal volume and an outlet port at another end thereof adapted for connection to a suitable vacuum pump.
The kit may further comprise a pump head having a pumping chamber including an inlet and an outlet, wherein said pump head is integrated with said first tissue cover, wherein said inlet is comprised in said first tissue cover and is in fluid communication therewith, and wherein said outlet is connectable to a powered drive apparatus comprising a drive unit coupled to a primary pump, in operation said powered drive apparatus being configured for operating said pump head via a non-mechanical coupling arrangement when driven by said drive unit and adapted for selectively providing a predetermined working pressure within said internal volume below ambient pressure of an external environment via operation of said pump head.
The kit may further comprise a venting arrangement for enabling venting of said internal volume to ambient when in use.
The kit is configured for being disposable.

According to this aspect of the invention, the kit in operation is assembled to form a device for facilitating treatment of damaged tissue, for example as per the second aspect of the invention.

According to a second aspect of the invention, a device is provided for applying a vacuum to a tissue, which may facilitate treatment of the tissue when this is damaged tissue, for example, the device, comprising:

(A) a first tissue cover defining an internal volume at least in cooperation with the tissue in operation of said device, said first tissue cover comprising first peripheral portion having a tissue facing contact surface adapted to be sealingly affixed to a first tissue portion circumscribing the tissue, said device adapted for enabling a vacuum to be selectively applied to said internal volume in operation of said device;

(B) a substantially contiguous layer of flowable sealing material configured for covering, in use of the device, at least a second tissue portion abuttingly adjacent at least a part of said first peripheral portion, said sealing material configured for filling gaps between said contact surface and said first tissue portion that may form leaks by flowing towards said contact surface under suction provided when said vacuum is applied to said internal volume, wherein said sealing material is uncoupled with respect to said contact surface at least prior to affixing said contact surface to said first tissue portion; and

(C) a second tissue cover covering at least a part of said layer.

The device may comprise one or more of the following features in any combination:
The substantially contiguous layer may be abuttingly adjacent the full periphery of the first peripheral portion, i.e., the layer fully circumscribes the first peripheral portion.
The contiguous layer may further cover an external portion of said first tissue cover adjacent said first peripheral portion.
The device may further comprise an interface comprising at least one of medication, lotions, gauze, bandages, packing material in said internal volume at lest in operation of said device.
The first tissue cover may comprise a first drape having a tissue-facing adhesive layer and a removable protective layer carried against said adhesive layer prior to use, and wherein said first peripheral portion is adapted to be affixed to the first tissue portion via said adhesive layer having first removed said protective layer. For example, the protective layer may comprise any one of a paper strip, tape and a film.
The second tissue cover may be configured for retaining said sealant material between said second tissue cover and said first tissue cover and for enabling said sealant material to flow to fill gaps as required during operation of said device.
The second tissue cover may comprise a second peripheral portion configured for affixing said second peripheral portion to a third tissue portion adjacent said second tissue portion. The second peripheral portion may be partially or fully circumscribing the second tissue portion.
The sealing material may comprise for example any one of petroleum jelly, vacuum grease.
The device is configured to be disposable.
The device may further comprise a venting arrangement for enabling venting of said internal volume to ambient when in use. For example the venting arrangement may comprise at least one bleeding orifice arrangement having an effective flow area compatible with enabling a desired flow rate to be provided during operation of the device. For example, the venting arrangement may be controllable and adapted for regulating, at least during operation of said system, the vacuum level at said internal volume; for example the controllable venting arrangement may comprise a pressure regulator or a solenoid valve.
The device may further comprise a port adapted for connection to an irrigation source for providing, at least during operation of said device, fluid communication between said internal volume and said irrigation source, such as to enable a desired irrigation material to be provided to the internal volume.

The device may have the following configurations:
In configuration (I), the first tissue cover may comprise a conduit having at one end thereof an inlet port opening into said internal volume and an outlet port at another end thereof adapted for connection to a suitable vacuum pump.
In configuration (II), the device may further comprise a pump head having a pumping chamber including an inlet and an outlet, wherein said pump head is integrated with said first tissue cover, wherein said inlet is comprised in said first tissue cover and is in fluid communication therewith, and wherein said outlet is connectable to a powered drive apparatus comprising a drive unit coupled to a primary pump, in operation said powered drive apparatus being configured for operating said pump head via a non-mechanical coupling arrangement when driven by said drive unit and adapted for selectively providing a predetermined working pressure within said internal volume below ambient pressure of an external environment via operation of said pump head.

According to the second aspect of the invention, a system is provided for applying a vacuum to a tissue. For example for treatment of tissue that is damaged tissue, comprising a device according to configuration (I) and variations thereof, and further comprising a vacuum source configured for generating a vacuum and applying said vacuum to said internal volume. The system may comprise one or more of the following features in any combination:
the vacuum source may comprise a vacuum pump head coupled to a drive unit, in operation said drive unit being configured for driving said pump head via a coupling arrangement whereby to selectively provide said vacuum in said internal volume. In at least some embodiments, the coupling arrangement is configured for enabling said pump head to be selectively engaged and disengaged from said drive unit in a manner not necessitating tools. Optionally, the pump head is disposable.
The system may further comprise a waste container defining a collection volume for collection of materials that may be drained from said tissue via said device during operation of said system, wherein said waste container is in fluid communication with an outlet port of said pump head. The waste container may be disposable.

According to the second aspect of the invention, a system is provided for applying a vacuum to a tissue, for example, for the treatment of tissue that is damaged tissue, comprising a device according to configuration (II) and variations thereof, and further comprising a powered drive apparatus comprising a drive unit coupled to a primary pump, in operation said powered drive apparatus being configured for operating said pump head via a non-mechanical coupling arrangement when driven by said drive unit and adapted for selectively providing said vacuum in said internal volume via operation of said pump head. The system may comprise one or more of the following features in any combination:
The pump head comprises an actuation chamber that is mechanically coupled to said pumping chamber, and wherein said non-mechanical coupling arrangement comprises a pneumatic coupling arrangement comprising a control volume of working fluid that pneumatically couples said primary pump with said actuating chamber, said mechanical coupling being configured for enabling said drive unit to operate said pump head by inducing pumping of said pumping chamber via pneumatic coupling between said primary pump and said actuation chamber.
The system further comprises a waste container defining a collection volume for collection of materials that may be drained from said tissue via said suction head assembly during operation of said system, wherein said waste container is in fluid communication with an outlet port of said pumping chamber. The waste container may be disposable.

According to a third aspect of the invention, there is provided a method for providing a self sealing enclosure for use over a target body part. The method may facilitate application of a vacuum to a target body part that is a tissue, for example for facilitating treatment of the tissue wherein the tissue is a damaged tissue. The method comprises:

(a) providing an enclosure comprising a device including a first tissue cover defining an internal volume at least in cooperation with the body part in operation of said device, said first tissue cover comprising a first peripheral portion adapted to be sealingly affixed to a first tissue portion circumscribing the targeted body part, said device adapted for enabling a vacuum to be selectively applied to said internal volume in operation of said device;

(b) placing said first tissue cover over a the body part,

(d) sealingly affixing said first peripheral portion to the first tissue portion;

(e) subsequently applying a flowable sealing material to cover in a substantially contiguous layer at least a second tissue portion abuttingly adjacent at least a part of said first peripheral portion, said sealing material configured for filling gaps between said first peripheral portion and said first tissue portion that may form leaks when a vacuum is applied to said internal volume; and

(f) covering at least a portion of said layer with a second tissue cover.

The method may comprise one or more of the following features in any combination:
In step (e) said substantially contiguous layer is abuttingly adjacent the full periphery of said first peripheral portion.
In step (d) said sealing material is further applied to an outside surface of said first tissue cover adjacent said first peripheral portion such that said contiguous layer extends to said outside of said first tissue cover. Optionally, the sealing material may also be applied to substantially the full said outside surface, wherein further to seal said outside surface.
The sealing material may be applied to a third peripheral portion of said outside surface adjacent said first peripheral portion.
Prior to step (b), at least one of medication, lotions, gauze, bandages, packing material may be provided.
In step (d), the second tissue portion fully or partially circumscribes said first peripheral portion, and thus sealing material can if desired, only be applied with respect to one or more parts of the peripheral portion where it is expected for leaks to occur.
The first tissue cover may comprise, for example, a first non-rigid drape having a tissue-facing adhesive layer and a protective layer carried against said adhesive layer prior to use, wherein prior to step (c) said protective layer is removed, and in step (c) said first peripheral portion is affixed to the first tissue portion via said adhesive layer. For example the protective layer may comprise any one of a paper strip and a film.
The second tissue cover may comprise a second peripheral portion and step (e) may further comprise sealingly affixing said second peripheral portion to a third tissue portion adjacent said second tissue portion. The second peripheral portion may be partially or fully circumscribing the second tissue portion.
The target body part may be a tissue, for example damaged tissue such as for example a skin burn or a skin injury.
The sealing material may comprise, for example, petroleum jelly or vacuum grease.

According to this aspect of the invention, there is provided a method for facilitating treatment of a target body part comprising a damaged tissue, said method comprising providing a self sealing enclosure for use over a target body part as disclosed.

According to this aspect of the invention, there is also provided a method for treating damaged tissue, comprising applying the aforesaid method for facilitating said treatment, and further comprising selectively applying a vacuum to said internal volume. Optionally, materials that may be drained from said damaged tissue may be collected via said device during operation thereof when a vacuum is applied to said internal volume. Optionally, these materials may be collected in a waste container, and these materials may be disposed with said waste container. The device can be removed from the body part after use, and disposed of.

It is to be noted that any features disclosed only for one of or two of the first or second or third aspect of the invention apply, mutatis mutandis, to one or more other aspects of the invention.

Thus, at least some embodiments of the present invention enable to overcome at least some of the difficulties often encountered in the prior art relating to sealably fixing a drape over a wound area, which often has irregular skin contours and which often results in voids being formed between the drape and skin in the prior art. When present, such voids form leaks and require higher suction flow rate to generate a vacuum to the desired level over a damaged tissue area, while many prior art vacuum pumps for generating a vacuum have limited flow.

At least some embodiments of the present invention avoid the need to apply various drape layers to ensure sealing, which need often arises in the prior art when attempting to cover a wound with a drape having a highly adhesive layer, which is difficult to handle and even more difficult to reposition onto the skin, and which results in many such voids being created, which are sealed in the prior art by the aforesaid plurality of overlapping drape layers.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 is a cross-sectional side view of a device according to a first embodiment of the invention; FIG. 1(a) and FIG. 1(b) respectively illustrate in fragmented cross-sectional side view details of the first and second peripheral portions of the embodiment of FIG. 1.

FIG. 2 is a cross-sectional side view of a device according to a variation of the embodiment of FIG. 1.

FIG. 3 is a schematic illustration of a system according to a first embodiment of the invention.

FIG. 4 is a schematic illustration of a kit for providing the embodiment of FIG. 1.

FIG. 5 is a cross-sectional side view of a device according to a variation of the embodiment of FIG. 1 or FIG. 2.

FIG. 6 is a cross-sectional side view of a device according to another variation of the embodiment of FIG. 1 or FIG. 2.

FIG. 7 is a cross-sectional side view of a device according to another variation of the embodiment of FIG. 1 or FIG. 2.

FIG. 8 is a cross-sectional side view of a device according to another variation of the embodiment of FIG. 1 or FIG. 2.

FIG. 9 is a cross-sectional side view of a device according to another variation of the embodiment of FIG. 1 or FIG. 2.

FIG. 10 is a schematic illustration of a system according to variation of the embodiment of FIG. 3.

FIG. 11 is a cross-sectional side view of a device according to a second embodiment of the invention.

FIG. 12 is a schematic illustration of a system according to a second embodiment of the invention.

### DETAILED DESCRIPTION OF EMBODIMENTS

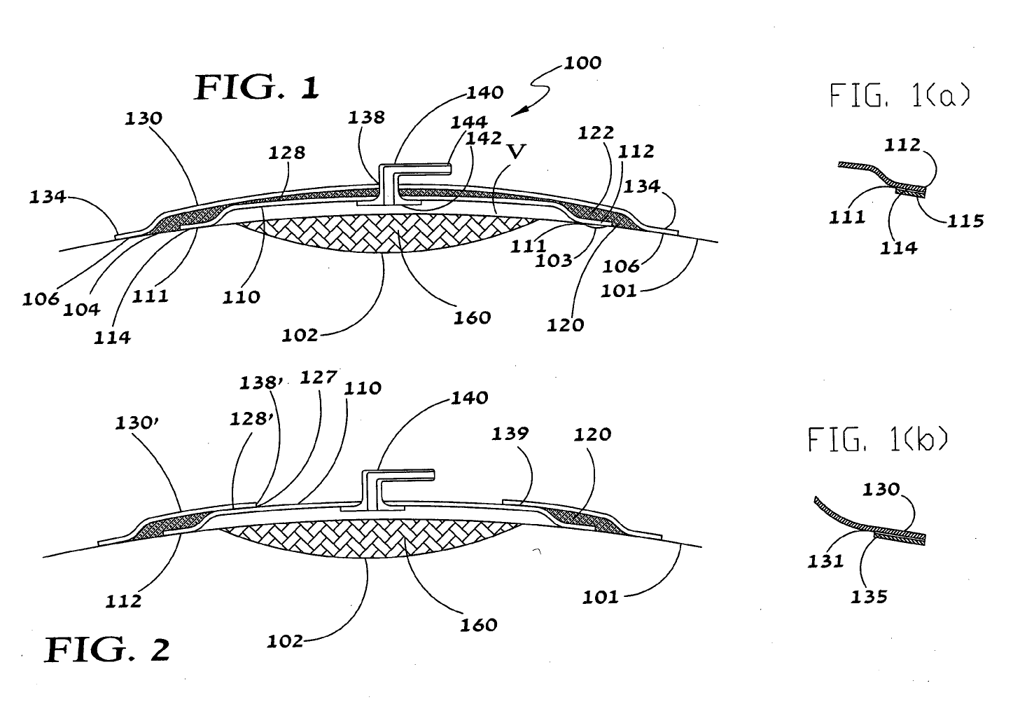

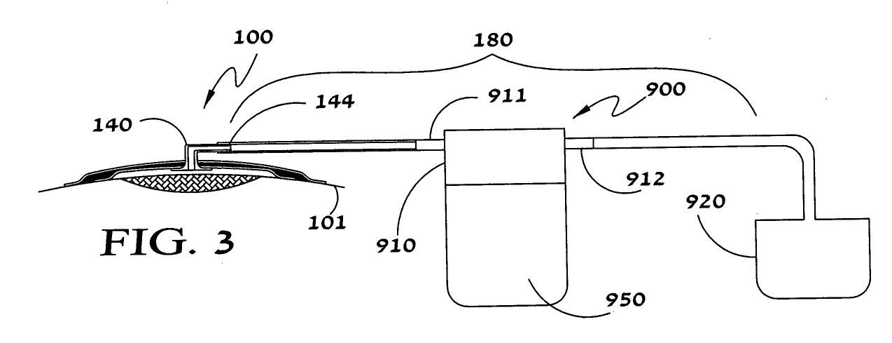

Referring to FIGS. 1 and 3, a device for applying a vacuum to a tissue, for example for facilitating treatment of damaged tissue, according to a first embodiment of the invention, generally designated 100, comprises a first, inner tissue cover 110, a substantially contiguous layer 120 of flowable sealing material 122, and a second, outer tissue cover 130. In the disclosed embodiments, the tissue to be covered (also interchangeably referred to herein as a target body part) is an external body tissue such as the skin and/or may include deeper body layers. Such a device is also referred to herein interchangeably as a tissue enclosure, wound enclosure or wound cover.

The first tissue cover 110 is in the form of a drape, typically non-rigid, having a shape and size sufficient to cover the damaged tissue 102 as well as a first tissue portion 103 of preferably healthy tissue (typically skin) circumscribing the damaged tissue 102. The first tissue cover 110 comprises a first peripheral portion 112 having a tissue facing contact surface 114 adapted to be sealingly affixed to a first tissue portion 103. The first tissue cover 110 is made of a suitable pliant material that facilitates conforming the first tissue cover 110 to the part of the body 101 onto which it is to be applied. Referring in particular to FIG. 1(a), the first tissue cover 110 comprises an adhesive layer 111 having a layer of adhesive that enables any part of the first tissue cover 110 that contacts the body tissue to be adhesively sealed in the area of mutual contact. The adhesive layer 111 is covered, prior to its use, with a protective layer 115, comprising a film, tape or strip of paper, for example, that is removed prior to adhering the first tissue cover 110 onto the tissue. The first tissue cover 110 may be obtained, for example, from a roll or standard-sized sheet of pliant material, already pre-coated with an adhesive layer and a protective layer, by cutting to size according to the particular geometry and size of the damaged tissue 102.

Having provided the first tissue cover 110 of the required shape and size, a suitable barrier member (not shown) may be provided and adhered to the generally central part of the first tissue cover 110 after the full protective layer 115 is removed, thereby effectively exposing only the tissue facing contact surface 114 to the tissue surrounding the damaged tissue, and again avoiding adhering the first tissue cover 110 directly to the damaged tissue 102. The barrier layer may comprise for example a screen material, such as open cell polyurethane foam, for example, which is inserted into the concavity of the wound to prevent tissue from growing above the body surface. Optionally, the barrier member may be non-porous and/or non-foam-like, for example including materials PVC (polyvinylchloride) or PVC free of DEHP (diethylhexyl phthalate, polyethylene, polyurethane, and so on. For example, the first tissue cover 110 may be made from an occlusive or semi-occlusive material which allows water vapor to permeate through, for example TEGADERM™ brand sealing film made by 3M Corporation, or OPSITE FLEXIGRID™ semi-permeable dressing made by Smith & Nephew.

As illustrated, the first tissue cover 110 is a single component in this embodiment. In alternative variations of this embodiment, however, the first tissue cover 110 may be formed from a plurality of strips of material, for example, which may be applied, in at least partial overlapping relationship with respect to one another, to the body part 101, together covering the damaged tissue 102.

In any case, the first tissue cover 110 may be made, for example, from any suitable medically compatible material, for example as used in the art for covering wounds burns and the like on the skin, and in particular as further used in conjunction with applying a sub-ambient pressure thereto.

In any case, a suitable damaged tissue interface may be provided for application to the damaged tissue, including for example any suitable medication, lotions, gauze, bandages, packing material, and so on, generally indicated at 160 in FIGS. 1, 2 and 4, and this may be applied directly to the damaged tissue, or provided with the first tissue cover 110, prior to the first tissue cover 110 being adhesively sealed onto the tissue portion 103.

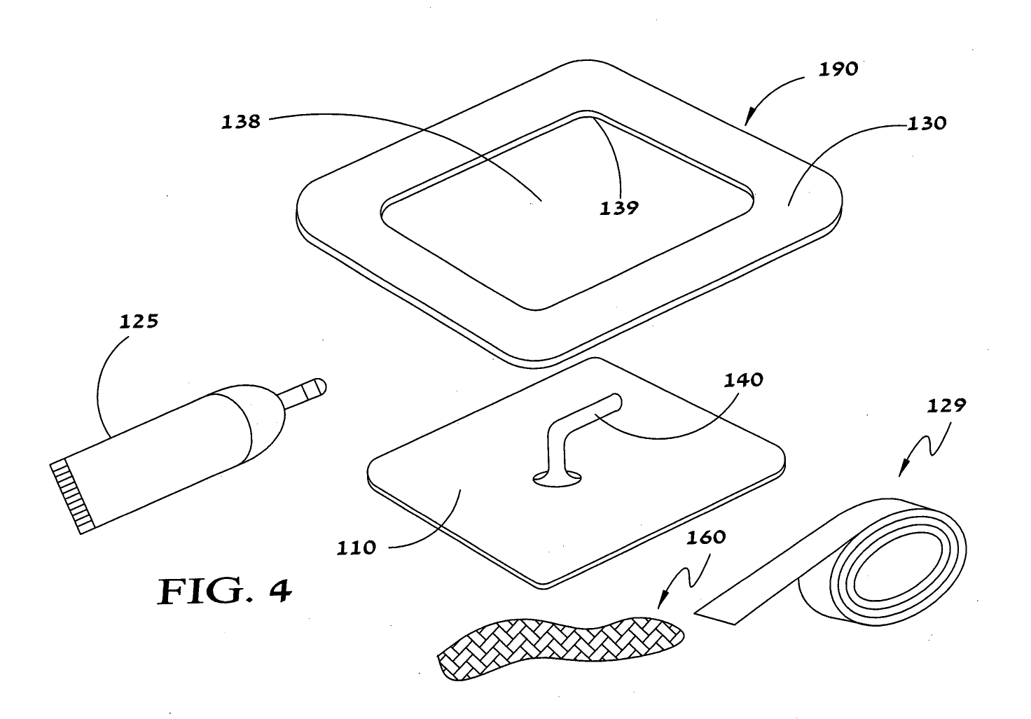

Furthermore, additional sealing between the first peripheral portion 112 and the first tissue portion 103 may be provided by sticking suitable adhesive tape 129 (FIG. 4) in overlapping relationship over the first peripheral portion 112 and the tissue adjacent thereto, which is thus considered herein to be part of the aforesaid first tissue portion 103, and correspondingly the parts of the adhesive tape adhered to this adjacent tissue are also considered herein to be part of the said first peripheral portion 112.

The first tissue cover 110 creates a sealed environment between the tissue cover 110 and the damaged tissue 103 in which a vacuum or negative pressure with respect to ambient can be maintained, and thus defines an internal volume V at least in cooperation with the damaged tissue 102 in operation of said device. For example, at least some of this internal volume V may be provided by the natural concavity of the damaged tissue 102 which is often present in damaged tissue, and/or, at least some of this internal volume V may be provided by forming the first tissue cover 110 with a concavity, and/or by manipulating the first tissue cover and adhering the same to the tissue so as to provide a concavity.

The device 100 is adapted for enabling a vacuum to be selectively applied to the internal volume V in operation of said device, and comprises at least one conduit 140 having at one end thereof an inlet port 142 open to and in fluid communication with the internal volume V, and an outlet end 144 being configured for connection to a suitable vacuum source 900 (FIG. 3).

The sealing material 122 is provided as a substantially contiguous layer 120 covering at least a second tissue portion 104 circumscribing and abutting adjacent the first peripheral portion 112. The sealing material 122 is a flowable, generally high viscosity fluid, and is configured for filling any gaps and plugging the ensuing leaks that may form between the contact surface 114 and the first tissue portion 103 as the device 100 is operated and a vacuum applied to the internal volume V, by flowing from layer 120 towards the contact surface under suction provided by the vacuum. In addition, such a material is medically compatible. Without being bound by theory, the size of such gaps to be filled may be determined by the amount and viscosity of material 122 that may flow thereinto during operation of the device. When each gap is filled, the flow of material 122 thereinto effectively terminates as the high viscosity of the material 122 requires a high pressure differential across the gap to continue flowing, which is not present in applications of the present invention. Small gaps between the contact surface 114 and the first tissue portion 103 at the same time provide a relatively large surface area of contact with the sealing material 122 with a relatively small flow cross-sectional area, reducing or preventing flow once the gap has been filled. The sealing material 122 is a non-putty, generally non-adhesive flowable liquid, though may be tacky, and while plugging leaks that may develop between the contact surface 114 and the first tissue portion 103, does not provide adhesion therebetween in the same manner as adhesive layer 111, and may even degrade the adhesive properties of said adhesive layer 111. When the material 122 is properly applied as disclosed herein, adhesion between the patient's tissue and the contact surface 114 via the adhesive layer 111 is already previously established. Thus the sealing material is uncoupled with respect to the adhesive layer 111 or the contact surface 114, i.e., the device is configured such that the sealing material is not in contact with the adhesive layer 111 or the contact surface 114, at least prior to adhesion of the contact surface to the first tissue portion 103. This uncoupling of the application of material 122 with the initial adhesion of the contact surface 114 to the first tissue portion 103 serves to avoid or minimize any migration and smearing of the material 122 onto the adhesive layer 111, prior to the initial adhesion of the contact surface 114 via the adhesive layer 111, which could otherwise form a barrier over at least part of the adhesive layer 111 preventing from the start that part of the adhesive layer 111 from being adhesively sealed to the tissue portion 103. Thus, the uncoupling feature also enables the contact surface 114 to be adhered to the first tissue portion 103 without interference from the material 122.

Such a material 122 may comprise, for example, a petroleum jelly, such as for example Vaseline, or a vacuum grease material such as for example Dow Corning 360 hydrophobic medical fluid lubricant for skin provided by Dow Corning. However, any other suitable high viscosity, medically compatible sealing material may be used, in the form of liquid, paste or gel, for example. On the other hand, highly adhesive, putty-like manually moldable materials are generally avoided, since their adhesive properties prevents flowing of such material from an outside of the peripheral portion 112 into gaps that may be required to be filled during operation of the device.

For example, the material 122 may be applied from a tube 125 (see FIG. 4) optionally with the aid of a suitable applicator, for example a sterile spatula, and dressed over an external portion 128 of the first tissue cover 110 adjacent the first peripheral portion 112, as well as over the second tissue portion 104, to provide a contiguous layer. In this embodiment, this external portion 128 constitutes substantially the whole of the outside of the first tissue cover 110, excluding the immediate entry point of the conduit 140. However, and as is illustrated in an alternative variation of this embodiment in FIG. 2, this external cover portion, indicated at 128′, may alternatively only constitute a part of the outside of the first tissue cover 110.

The sealing material 122 is applied after the first tissue cover 110 is adhesively sealed to the tissue, and thus the sealing material is uncoupled with respect to the contact surface at least prior to affixing the contact surface to the first tissue portion.

The second tissue cover 130 is configured for covering at least the sealant layer 120 after this has been applied, and comprises a second peripheral portion 134 configured for affixing said second peripheral portion to a third tissue portion 106 circumscribing and adjacent the second tissue portion 104. The second tissue cover 130 may be substantially as disclosed for the first tissue cover 130, mutatis mutandis, the main difference being that the second tissue cover 130 is substantially larger, and furthermore comprises an opening to 138 through which the conduit 140 passes. The second tissue cover 130 does not have to be, though may be, adhesively sealed against the third tissue portion 106, and it is often sufficient to affix the tissue cover 130 onto the third tissue portion 106. The second tissue cover 130 is thus configured to retain the sealing material 122 in a reservoir formed in the space between the first tissue cover 110 and the second tissue cover 130, so that the material is available to plug gaps that may form between the peripheral portion 112 and the first tissue portion 103, and does not instead migrate away from the area where leaks are likely to occur. Thus, for example, and referring to FIG. 1(b), the second tissue cover 130 may also comprise an adhesive layer 131 including a layer of adhesive that enables any part of the second tissue cover 130 that contacts the body tissue or the outside 128 of the first tissue cover 110 to be adhesively sealed in the area of mutual contact. The adhesive layer 131 may also be initially covered with a protective layer 135, comprising a film, tape or strip of paper, for example, that is removed prior to adhering the second tissue cover 130 onto the tissue portion 106. The second tissue cover 130 may thus also be obtained, for example, from a roll or standard sized sheet of pliant material, already coated with an adhesive layer and a protective layer, by cutting to size to be sufficiently greater than the first tissue cover 110 to enable superposition overt the second tissue portion 104 and the third tissue portion 106, and may be made from the same materials.

In use, the protective layer 135 is removed, and the second tissue cover 130 is placed over the first tissue cover 110 including the sealant layer 120, and adhesively sealed onto the third tissue portion 106. As with the first tissue cover 110, mutatis mutandis, sealing tape 129 may optionally be used for facilitating sealing of the second peripheral portion 134, and may also be applied over the sealing material 122 of the sealant layer 120.

In alternative variations of this embodiment, the second tissue cover may be configured for partially covering the sealant layer 120 (after this has been applied), and the respective second peripheral portion is configured for affixing the second peripheral portion to a respective third tissue portion that partially circumscribes and is adjacent to the second tissue portion 104.

The first tissue cover 110 and the second tissue cover 130 may each be made from an impermeable material, or from a semi-permeable material that allows limited gaseous exchange therethrough. In the latter case, it is possible to effectively seal the outside of the first tissue layer 110 by coating the full extent or most thereof with said sealant material 122, for example as in the embodiment of FIG. 1.

In operation, once the device 100 is adhesively sealed onto the body portion 101, if any leaks develop between the peripheral portion 112 and the tissue portion 103, the material 122 flows to plug the leak by means of the suction generated by the vacuum in the internal volume V. After use, the device 100 may be removed from the body portion 101 in any number of suitable ways. For example, the device may de destructively removed, or forced off the body part with the application of force higher than is normally experienced by the device during usage.

In the variation of the first embodiment illustrated in FIG. 2, an inner peripheral portion 139 of the second tissue cover 130 is also adhesively sealed onto the outside of the first tissue cover 130, over an area 127 not covered by the annular sealant layer, and thus may optionally, though not necessarily, comprise a relatively enlarged opening 138′, compared with the embodiment of FIG. 1.

In other variations of the first embodiment, the first tissue cover and/or the second tissue cover may be formed as a relatively rigid or semi rigid structure, for example made from rigid or semi rigid materials.

Referring to FIG. 4, the device 100 may be provided as a kit 190, comprising the first tissue cover 110, the sealant material 122 comprised in a suitable container such as a tube 125, and the second tissue cover 130, and optionally sealing tape 129 wound packing materials indicated at 160, and applicator (not shown).

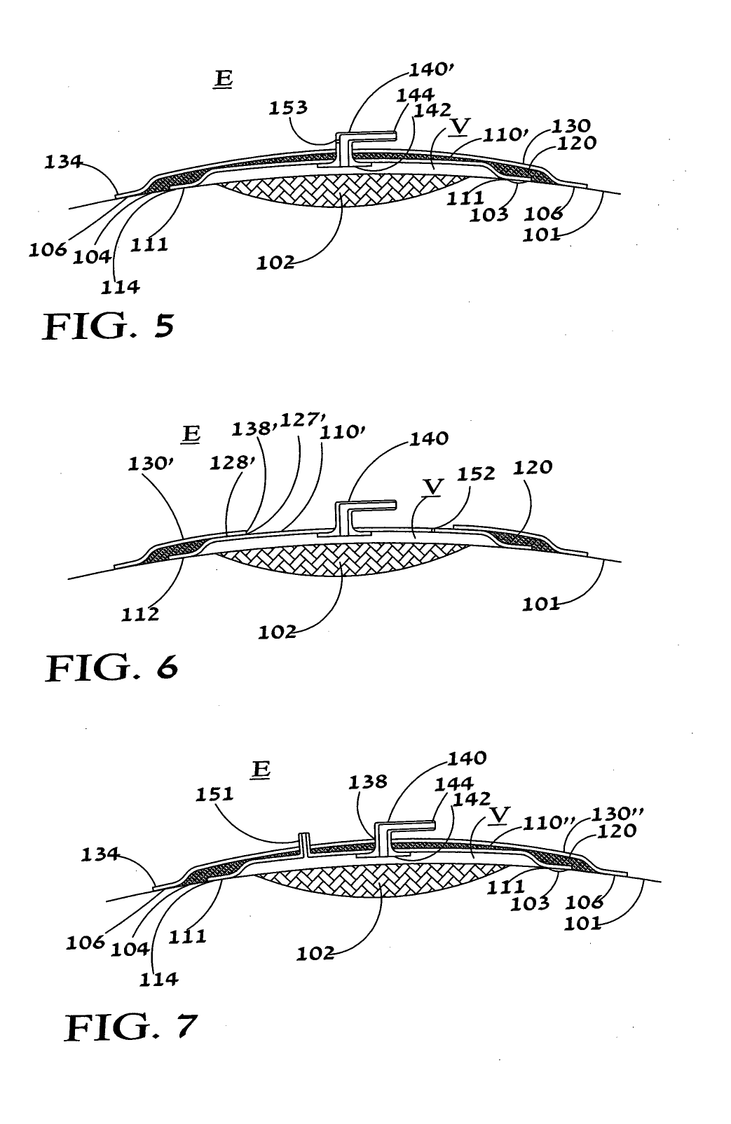

A number of variations of the first embodiment of the device is illustrated in FIGS. 5 to 9, each variation being as disclosed for the first embodiment, mutatis mutandis, but with the respective variations, as disclosed below. In each of FIGS. 5 to 9, similar components to those of the first embodiment have the same reference numerals with respect thereto.

Referring to FIGS. 5 to 7, a venting arrangement is provided to the device, including a bleed hole, or other orifice, which creates a predetermined, controlled pressure drop across it as external ambient air flows through the bleed holes and towards the vacuum source during operation of the device. Such bleed holes are disclosed, for example in WO 2007/088530, WO 2007/013064 and WO 2007/013049 to the present inventor, the contents of these publications being incorporated herein in their entirety. In operation, the flow level will generate the desired vacuum level across the bleed hole, which when in fluid communication with the internal volume will provide such vacuum control at the internal volume V. When air flows through such a bleed hole, which restricts the flow, a pressure differential needs to be generated across such hole to force the flow therethrough. To increase the flow requires an increase in the pressure differential, and by changing or controlling the flow through the bleed holes (by controlling the flow by the vacuum source), the pressure differential can be controlled to any desired level. Since one side of the bleed hole is at ambient pressure, the generated pressure differential across such bleed hole provides the sub-ambient pressure on the other side of such bleed hole, corresponding to a particular setting of the vacuum source. The bleeding hole(s) may be used for venting the internal volume and/or for preventing blockages and/or removing any blockages (including fluids, coagulates, exudates, etc.) in the conduits between the device and the vacuum source, as well as for assisting in the process of moving exudates within the conduit 140 towards the outlet port 144 thereof and typically to a waste container.

Referring to FIG. 5, a modified conduit 140′ is provided, similar to conduit 140 of the embodiment of FIG. 1, mutatis mutandis, but comprising at least one bleeding hole 153 of predetermined effective area in close proximity to the internal volume V and thus to the second tissue cover 130, and in direct communication to the external environment E, such that ambient air can enter the conduit 140 and flow together with the exuded fluids when negative pressure is present. Ambient pressure may be restored in the internal volume V when the vacuum source is not operating. The bleeding hole may be a calibrated orifice, providing for controlled flow of ambient air into the internal volume V. In alternative variations of this embodiment bleeding hole may comprise other flow restrictors, for example, a hole plugged with open cell foam or an open pore sintered metal plug, which restrict the flow, but are not susceptible to plugging as is a small orifice. Alternatively, the “bleeding” feature may be provided by the tissue cover 110′ itself when this is made from a semi-permeable material, which while not in all cases providing flow control nevertheless may provide advantages of venting, facilitating movement of exudates along conduit 140, and so on.

Additionally or alternatively, one or more bleeding holes 152 may be provided in the first tissue cover 110′, as illustrated in the embodiment of FIG. 6, in which the first tissue cover 110′ is similar to that disclosed for the embodiment of FIG. 1, mutatis mutandis, but modified to include the one or more bleed holes 152. The embodiment of FIG. 6 is based on the variation of the first embodiment illustrated in FIG. 2, wherein the sealant layer extends over a part of the outside of the first tissue cover 110′, and the second tissue cover 130′ comprises an enlarged, generally central opening 138′, exposing a central part 127′ of the first tissue cover 110′ to the external environment. The one or more bleeding holes 152 are formed in the central part 127′, and operate in a similar manner to that described for the embodiment of FIG. 5, mutatis mutandis.

In a variation of the embodiment of the embodiment of FIG. 6, and referring to FIG. 7, the sealant layer 120 and a second tissue cover 130″ substantially cover most or the entire outside of the first tissue layer 110″, in a similar manner to the embodiment of FIG. 1, mutatis mutandis, but modified to enable include the one or more bleed holes in the form of short tubes 151 that penetrate through the second tissue cover 130″, sealant layer 120, and the first tissue layer 110″ to provide controlled fluid communication between the internal volume V and the external environment E via the tubes 151. Such tubes may be provided after the device has been fully installed on the body part 101. Instead of tubes, though, aligned holes may be provided in the first tissue layer 110″ and the second tissue layer 130″, which are sealingly joined together at each pair of corresponding matched holes to enable communication between the internal volume V and the outside environment without passing through the sealant layer itself.

Optionally, a vent may be provided, for example in the form of a tube coupled at one end thereof to each respective bleed hole for each of the embodiments illustrated in FIGS. 5 to 7, to enable the device to be vented to a remote location via the second end of the vent tube.

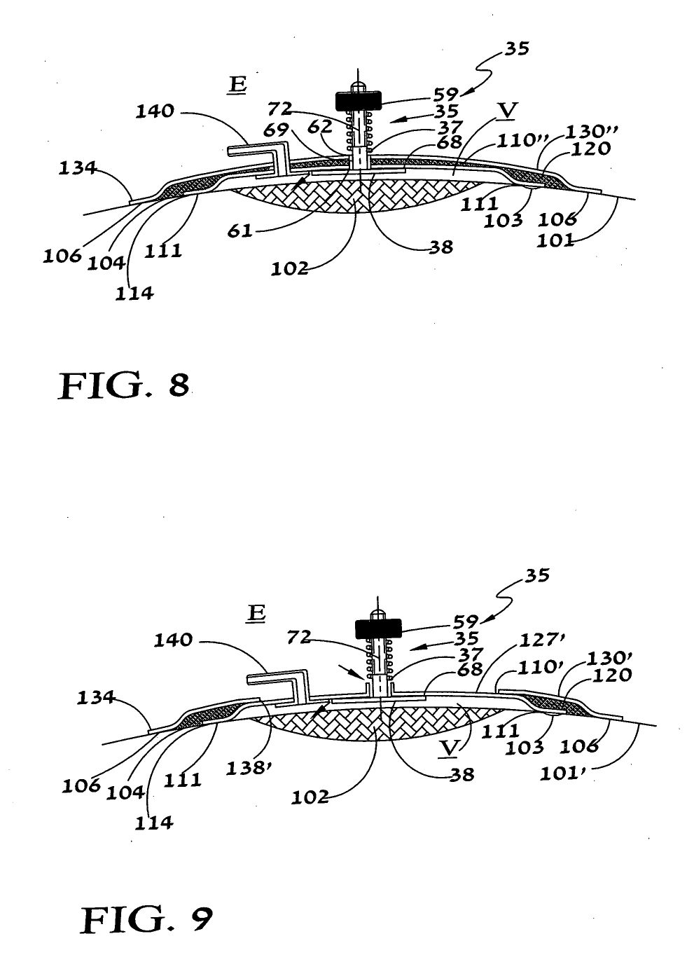

Referring to FIG. 8, in another variation of the first embodiment, a pressure regulator 35 may be provided to the device, comprising a venting valve arrangement adapted for enabling ingress of external ambient air into the system responsive to a reduction in said working pressure below a predetermined datum pressure with respect to said external ambient pressure and for discontinuing said ingress when said datum pressure is restored. The venting valve arrangement may comprise, for example, a venting valve arrangement as disclosed in the aforementioned WO 2007/013064, and comprises an inlet port having a valve seat in selective fluid communication with said ambient air, and a valve seal biased for sealingly closing with respect to said valve seat by means of a resilient element generating a biasing force of magnitude substantially less than and in a direction generally opposed to a pressure-induced force acting on said valve seal when said working pressure is less than said datum pressure.

In particular, a small length of tubing 69, having an inlet 61 and an outlet 62, is provided penetrating through the second tissue cover 130″, sealant layer 120, and the first tissue layer 110″ to provide a conduit between the internal volume V and the external environment E. The pressure regulator 35 comprises a vent valve arrangement having a valve seal 38 that cooperates with valve seat 68 in the inside surface of the first tissue layer 110″ for providing sealing engagement with inlet 61 when the regulator 35 is in the closed position. The valve seal 38 is mounted on a pin 72 having a nut 59 thereon, the axial position of which relative to the pin is adjustable. The pin 72 is reciprocably movable within the tubing 69 and the coaxial lumen of a helical spring 37, located between the nut and the outlet 62, between an open position in which the seal 38 is displaced from the seat 68, and the said closed position. The regulator 35 is urged to the open position when there is a pressure difference between the ambient air pressure of the external ambient environment E and the pressure within the internal volume V that exceeds a threshold value M. When this pressure difference is at or less than the threshold value M, the regulator 35 is urged to the closed position by means of the restoration force of the spring 37. The datum restoration force provided by spring 37 can be adjusted by means of nut 59, in order to control the threshold value M, and thus the vacuum conditions in the internal volume V at which the regulator 35 opens to the external environment E.

In the embodiment illustrated in FIG. 8, the pressure regulator 35 may be mounted to the device at any suitable position, though typically at a position where operation thereof will be unhindered by other equipment or parts of the patient's body, or where operation thereof will not be compromised by specific treatments that may be needed to be administered to the patient. Alternatively, the regulator 35 may be mounted in a suitable conduit, for example at a first end of a conduit that has its second end fixed to, and in open communication with, the outlet 62, and such an arrangement may be useful in cases where the area directly over the enclosure is unsuitable, for example where the patient is covered with blankets which are also draped over the enclosure.

Optionally, the regulator 35 may also comprise a biological or other suitable filter to prevent possible contamination of the wound via the confined volume V, and/or possible contamination of the external environment E.

In a variation of the embodiment of the embodiment of FIG. 8, and referring to FIG. 9, the sealant layer 120 extends over a part of the outside of the first tissue cover 110′, and the second tissue cover 130′ comprises an enlarged, generally central opening 138′, exposing a central part 127′ of the first tissue cover 110′ to the external environment. The regulator can be formed in the central part 127′, and operate in a similar manner to that described for the embodiment of FIG. 5, mutatis mutandis, with the difference that the small tubing 69 may be replaced with a simple opening in the central part 127′ with respect to which the regulator 35 may be mounted. Of course, the regulator may instead be mounted in a position on the device wherein there is the second tissue cover 130′ and optionally part of the sealant layer 120 as well, in a similar manner to that disclosed for the embodiment of FIG. 8, mutatis mutandis.

The device 100 according to the first embodiment or any variation thereof is formed as a disposable item, made from disposable materials, to be disposed of after use.

The device 100, according to the first embodiment or indeed any variation therefore as disclosed herein, may be used in a system for treating damaged tissue, in conjunction with a suitable vacuum source. Thus, a first embodiment of such a vacuum system, designated herein with reference numeral 180 and referring to FIG. 3, comprises a suitable vacuum source 900 and device 100, and the conduit 140 may be operatively connected to the vacuum source 900 for selectively enabling a suitable vacuum to be set up in the internal volume V when the device 100 is adhesively sealed onto the desired body portion 101. The system 180 further comprises a waste collection container 920 for collection of exudates and other fluids or materials that may flow from the internal volume V through conduit 140 when a vacuum is applied to the internal volume V.

In this embodiment, the vacuum system 180 comprises a suction or vacuum pump apparatus or system, for example as disclosed in any one of the aforementioned publications WO 2007/088530, WO 2007/013064 and WO 2007/013049, and thus comprises a pump head 910 coupled to a powered drive 950 for driving the pump head, and the device 100 is connected to the pump head in a similar manner to the enclosure disclose in the aforesaid publications, mutatis mutandis. The pump head 910 comprises a reciprocating pump, such as for example a piston pump or a diaphragm pump, including pumping chamber, driven by the pump drive 950, a pump inlet 911 to which the conduit 140 is directly or indirectly coupled, and a pump outlet 912 coupled to the collection container 920, and in operation, exudates and other material is transported from the internal volume V to the waste container via the pumping chamber. The pump head 910 is a disposable part, and is configured for being releasably attachable to the drive 950, which is a reusable part, in a simple manner not requiring tools. In alternative variations of this embodiment, the pump head may be releasably attached to the drive in a manner requiring tools. In these or other variations of this embodiment, the pumping apparatus comprises a powered peristaltic pump arrangement rather than a reciprocation pump. In these or other variations of this embodiment, the waste container 920 may be omitted, and any exudates can be discharged from the pump head 910 directly to a sink or drain; alternatively, such variations of the embodiment without the container may be useful when treating damaged tissue that does not contain exudates.

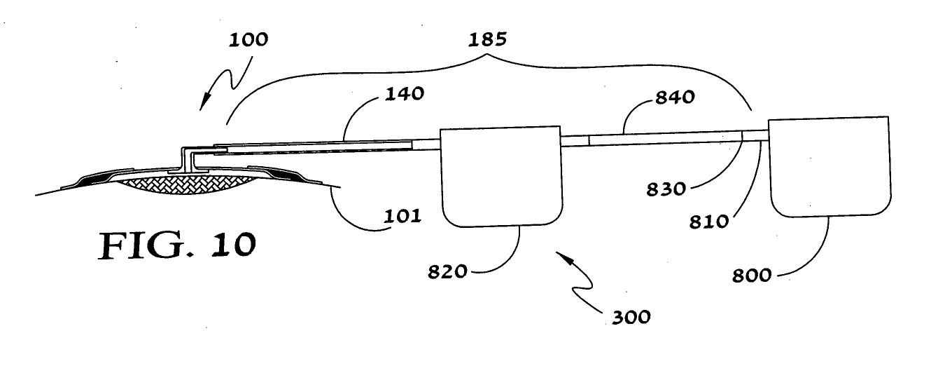

Referring to FIG. 10, a second embodiment of the system of the invention, designated 185, also comprises a device 100 as disclosed herein for the embodiment including any variation thereof (although the device according to the variation of FIG. 2 is illustrated), and a vacuum source 800 having at least an inlet 810 operatively connected to the conduit 140 via a waste container 820. The vacuum source 800 comprises an in-house suction source such as for example a hospital wall suction system, though alternatively, the vacuum source 800 may comprise any other suitable pump capable of providing the required vacuum. A suitable biological filter 830 is provided downstream of the container 820 to prevent cross-contamination between the device 100 and the vacuum source 800 during operation of the system 300.

The container 820 is also disposable, and together with device 100 and with a length of tubing 840 for connection of the container to the vacuum source and including the biological filter 830, may be disposed of after use.

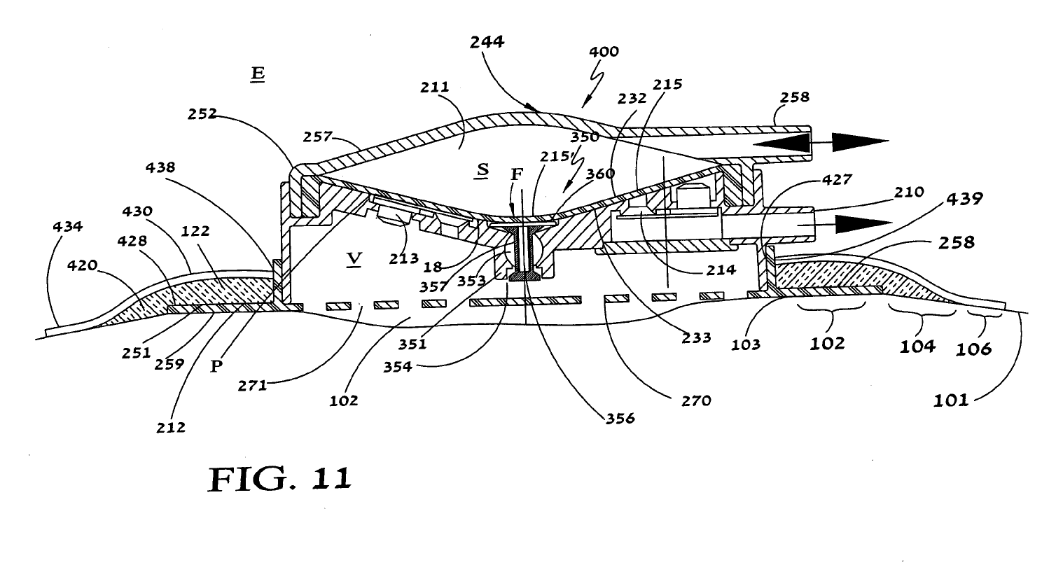

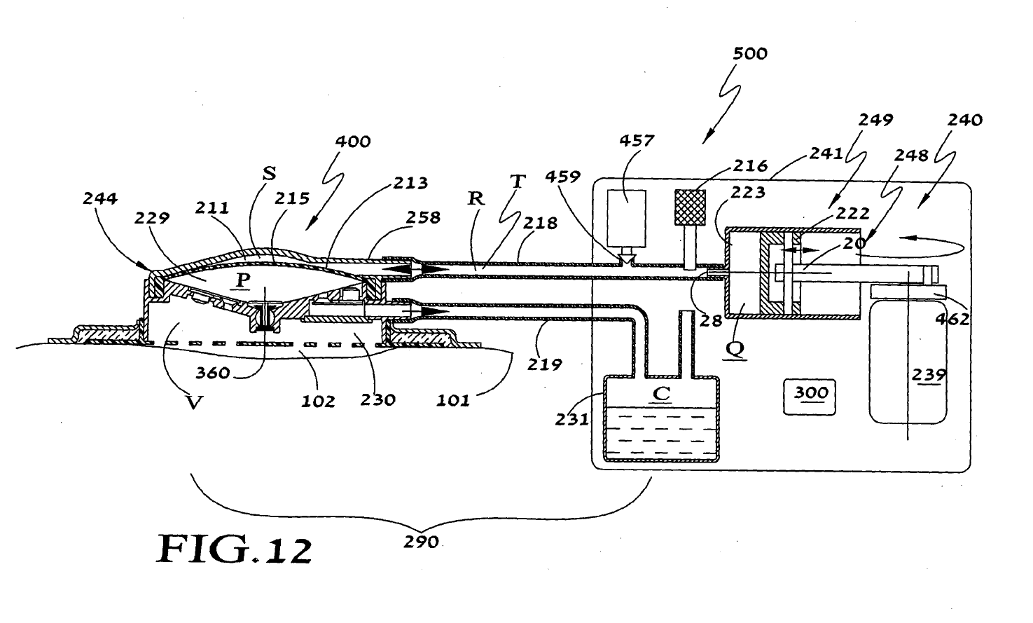

A device for facilitating treatment of damaged tissue according to a second embodiment of the invention, generally designated 400, is illustrated in FIGS. 11 and 12, and comprises all the elements and features of the first embodiment of the device, mutatis mutandis, with the main difference that in the second embodiment, the device 400 comprises, in addition to a first, inner tissue cover 212, a substantially contiguous layer 420 of said flowable sealing material 122, and a second, outer tissue cover 430, also a pump head 244 integrated with said first tissue cover 212. The device 400 may be used as part of system 500, illustrated in FIG. 12, for providing a vacuum at the damaged tissue 102, for example in a similar manner to the system and method for providing suction, and the suction head assembly, as disclosed in co-pending International Patent Application PCT/IL2008/000624 to the present inventor, published as WO 2008/135997, and the contents of this application are incorporated herein in their entirety.

The first tissue cover 212 comprises an open end 230. The slave pump head 244 is in the form of a dual chambered diaphragm type pump head, having a substantially rigid or semi rigid casing 252 comprising a first part 256 and a second part 257, and a barrier member 215 essentially dividing the working volume of the casing 252 into a pumping chamber 229 and a juxtaposed actuation chamber 211. The first part 256 comprises a valved pump inlet port 213 and a valved pump outlet port 214, each provided with suitable one-way valves. The barrier member 215 is sealingly connected at its periphery to the wall of casing 252, and comprises a pump chamber facing surface 233 and an actuation chamber facing surface 232. The arrangement is such as to ensure fluid flow in one direction through the pump head 244 from inlet port 213 to outlet port 214 via pumping chamber 229.

The pump head 244 is configured for enabling pumping of fluids therethrough and the barrier member 215 is substantially impervious to the fluids being transported through the pump head 244 during operation thereof, and acts as a pumping member. The pumping chamber 229 provides a variable pumping volume P, the barrier member 215 being reversibly deformable and/or movable to provide any desired position between: a first position in general contact or close proximity to the rigid part 256, to define a nominal minimum pump volume and a second position, maximally spaced from the rigid part 256 during operation of the system, to define a maximum pump volume. At the same time, the actuation chamber 211 comprises a variable volume S that varies in inverse relationship with respect to pumping volume P. Also, barrier member 215 provides the function of isolating the pumping chamber 229 from the actuation chamber 211, such as to prevent any fluid communication or contamination therebetween, and is also configured for responding to changes in pressure between the pump chamber facing surface 233 and the actuation chamber facing surface 232 thereof such as to equalize the pressures acting on the surfaces 232 and 233 of the barrier member 215.

Thus, in pumping mode, the pump head 244 provides a pumping action by attempting to equalize the pressures acting on either surfaces 232 and 233 of the barrier member 215, i.e., between the two chambers 211, 229, by changing the relative magnitudes of the volumes thereof. A pulsating pressure pulse may be set up in the actuating chamber 211, by means of the system 500 via pneumatic coupling therebetween, which in turn drives the pumping action of the pump chamber 229.

The device 400 further comprises a vent valve 350 for selectively allowing and preventing fluid communication between the internal volume V and the outside environment E by enabling selectively venting therebetween. Valve 350 comprises a valve housing 351, having a valve outlet 354 to volume V, a valve inlet 353 in fluid communication with an outside of the device 400. The valve 350 comprises an actuable member 356 cooperating with said housing 351, biased in the closed position preventing fluid communication between the pumping chamber 229 and the valve inlet 353 until acted upon by an actuator. The actuator is in the form of a paddle 360 having a bar that projects into, and is accommodated in, a well formed in the valve stem 357, which is made from a resilient material, such as for example rubber. The barrier member 215 may be selectively used at the end of the pressure stroke also for mechanically actuating the valve 350 by further increasing the pressure in the actuating chamber, and thus enables to controllably vent the first tissue cover 212. At least a part 215′ of the barrier member 215 is displaced and/or translated and/or deformed beyond that required for providing the nominal minimum pump volume so as to apply a mechanical force to the valve stem 357 via the paddle 360, which results in stretching of the valve stem 357, and opening of the valve, and opening of the valve 350 thus occurs at a nominally constant minimum pump chamber volume, and while no actual pumping is taking place. During regular pumping mode of said system 500, when the valve 350 is in the closed position, the internal volume V is substantially sealed from the external environment E.

Optionally, one or more ports (not shown) may be provided at the enclosure 212 for one or more functions. For example, one such port may be provided for enabling the pressure thereat to be monitored directly, for example by connecting thereto a suitable pressure transducer. Optionally, another such port may be provided for connection to an irrigation source for selectively providing an irrigation fluid to the target volume.

The first tissue cover 212 has a peripheral portion in the form of a flexible outer rim or flange 251 that is sealable to the first tissue portion 103 on periphery of the damaged tissue 102, via contact surface 259. The flange 251 may be made of a compliant material, such as for example polyurethane, which facilitates conforming of the flange 251 to the perimeter of the body part comprising the damaged tissue. Optionally, the lower rim or contact surface 259 of the flange may comprise an adhesive coating, which prior to use may be covered with a protective covering such as a suitable removable tape or film for example, for facilitating application and sealing of the rim to the first tissue 103. Furthermore, the first tissue cover 212 comprises a screen 270 at or near the open end 230, the screen 270 having a plurality of openings 271 configured for allowing adequate fluid communication between the damaged tissue 102 and the cover 212, while preventing solid matter such as large particles that may be present at the target area, i.e. the damaged tissue 102, from being suctioned by the system 500 during operation thereof, and thus minimize the risk of the valves and/or pumping chamber 229 becoming clogged with such matter. In this embodiment, the flange 251 and screen 270 are in the form of a unitary body, which is adapted for being mounted to the enclosure 212 by any suitable means, for example, heat welding, bonding, and so on, but in alternative embodiments alternative constructions may be provided.

Alternatively, and in other variations of this embodiment, the screen 270 may be omitted, and in any case a suitable interface may be provided in volume V, for example comprising one or more of any suitable medication, lotions, gauze, bandages, packing material, and so on, for application to the damaged tissue. The interface may be applied directly to the damaged tissue, or provided with the first tissue cover 212, prior to the first tissue cover 212 being adhesively sealed onto the tissue. In these or other alternative variations of this embodiment, the valve 350 may be omitted, and optionally one or more bleeding holes, and/or a pressure regulator, may be provided, in a similar manner as disclosed for the first embodiment of the device and variations thereof, mutatis mutandis.

The device 400 further comprises sealant layer 420 of said sealing material 122, as already disclosed for the first embodiment and variations thereof, mutatis mutandis. The sealant layer 420 is also applied after the flange 251 is adhesively sealed onto the first tissue portion 103, and covers a second tissue portion 104 and the outside 258 of the flange 251 in a contiguous manner.

The sealing material 122 is applied after the first tissue cover 212 is adhesively sealed to the tissue, and thus the sealing material is uncoupled with respect to the contact surface at least prior to affixing the contact surface to the first tissue portion.

A second tissue cover 430 is configured for covering at least the sealant layer 420 after this has been applied, and comprises a second peripheral portion 434 configured for sealingly affixing said second peripheral portion to a third tissue portion 106 circumscribing and adjacent the second tissue portion 104. The second tissue cover 430 may be substantially as disclosed for the embodiment of the device or variations thereof, mutatis mutandis, the second tissue cover 430 comprising an opening 438 through which the pump head 244, or at least ports 258 and 210, projects. The second tissue cover 430 also comprises an adhesive layer having a layer of adhesive that enables any part of the second tissue cover 430 that contacts the body tissue or the outside 428 of the first tissue cover 212 to be adhesively sealed in the area of mutual contact. The adhesive layer is initially covered with a protective layer, comprising a film or strip of paper for example, that is removed prior to adhering the second tissue cover 430 onto the tissue portion 106. In use, the protective layer is removed, and the second tissue cover 430 is placed over the first tissue cover 212 including the sealant layer 420, and adhesively sealed onto the third tissue portion 106, and sealing tape may also optionally be used for facilitating sealing of the second peripheral portion 434. As with the first embodiment, mutatis mutandis, the second tissue cover 430 may be affixed in place without sealing onto the third tissue portion 106, and is configured for containing the sealant material 122 and for enabling the same to be used for plugging leaks that may form between the first tissue cover 212 and the body part 101.

As with the first embodiment, the material 122 may be applied and dressed over an external portion 428 of the first tissue cover 212 abuttingly adjacent to the flange 251, as well as over the second tissue portion 104. In this embodiment, this external portion 428 constitutes a part of the outside of the first tissue cover 212, while preventing interference with at least the ports 258 and 210. The second tissue cover 430 comprises an inner peripheral portion 439 that is configured for being adhesively sealed onto the outside of the first tissue cover 212, over an area 427 not covered by the generally annular sealant layer 420.

In this embodiment, the second tissue cover 430 and the first tissue cover 212 (or at least the flange 434 thereof) is generally compliant such that it may conform to the shape of the surface of the body part 101 on which it is to be used.

As with the first embodiment and variations thereof, mutatis mutandis, the device 400 may also be provided as a kit, comprising the slave pump head 244 integrated with a first tissue cover 212, the sealant material 122 comprised in a suitable container such as a tube, and the second tissue cover 430, and optionally sealing tape and wound packing materials.

Referring to FIG. 12, system 500 comprises, in addition to device 400, suitable means configured for operating said pump head via a non-mechanical coupling arrangement. In a first embodiment of the system, said means comprises a powered pump apparatus 240 comprises drive unit 239 and primary pump 249. The drive unit 239 is operatively connected to a system 300 for controlling at least one operative parameter of the motor 239, is accommodated in housing 241, and comprises a powered drive, such as an electric motor, provided with power from an electric mains or battery (not shown), for example. Optionally, the speed and/or direction of motion provided by the motor may be variable, for example the motor 239 may be a stepper motor, and may comprise a flywheel 462.

A reciprocating mechanism 248, is provided for converting the rotary drive of the motor 239 to reciprocating motion of rod 20, although in other variations of the first embodiment of the invention, the powered drive of the powered pump apparatus 240 may comprise a linear motor, for example, for providing reciprocating motion optionally controllably variable in speed.

The drive unit 239 is coupled to primary pump 249, which in this embodiment is in the form of a piston pump (although in variations of this embodiment, a diaphragm pump may be used instead), to provide a master pump chamber 223 having a variable pumping volume Q, and the second part 257 of device 400 comprises a port 258 that provides fluid communication between the actuation chamber 211 and the powered pump apparatus 240, in particular the master pump chamber 223, via a conduit 218 or the like having internal volume R.

The pumping volume Q, volume S and the internal volume R together define a control volume T of working fluid that couples the piston 222 with the barrier member 215. Typically, the working fluid is air, and thus the control volume T pneumatically couples the master pump chamber 223 with the actuation chamber 211.

Thus, as the motor 239 operates and drives reciprocation of the piston 222, a corresponding pulsating flow is set up in the control volume T, alternately increasing and decreasing the volume of the actuation chamber 211, which in turn respectively decreases and increases the volume P of slave pump chamber 229, providing a periodic suction force in the volume V via the first tissue cover 212.

The control volume T is in fluid communication with at least one pressure sensor or transducer 216, or other suitable air pressure measuring device, for monitoring the pressure therein, operatively connected to the control system 300.

In the illustrated embodiment, a waste container 231 is accommodated in housing 241, and may be selectively removed therefrom for disposal, for example, optionally while still connected to the device 400 via conduit 219 coupled to exit port 210. The container 231 defines a collection volume adapted for collecting waste materials, particularly liquids and other flowable materials, from the damaged tissue 102 to which the system 500 is coupled. Concurrently with removal of the container 231 is the disconnection of conduit 218 from the drive unit 240. In alternative variations of the embodiment, the waste container 231 may be attached to the housing 241, or alternatively unattached thereto, and in any case positioned at any point between the pump head 244 and the housing 241.

A controllable pressure venting arrangement may be provided, comprising a solenoid 457 coupled to a valve 459 that serves to selectively vent the control volume T to the atmosphere when desired. For example, the controllable pressure venting arrangement may be used for synchronizing pump head 244 with the primary pump 249. Alternatively, such synchronization may be achieved by providing a controlled leakage outlet in the master pump chamber 223, which may also allow for controlling the pressure induced at the actuation chamber 211.

Thus, a number of embodiments have been disclosed, in which a device is provided for providing a vacuum to a tissue, for example for facilitating treatment of tissue that is damaged tissue, including: a first tissue cover for covering the damaged tissue and having first peripheral portion with a tissue facing contact surface adapted to be sealingly affixed to a first tissue portion surrounding the damaged tissue, the device being adapted for enabling a vacuum to be selectively applied to the damaged tissue in operation of said device; a substantially contiguous layer of flowable sealing material configured for covering a second tissue portion abuttingly adjacent at least a part of the first peripheral portion, wherein the sealing material is configured for filling gaps between said contact surface and said first tissue portion that may form leaks, by flowing towards said contact surface under suction provided when said vacuum is applied to the damaged tissue in operation of the device, and wherein said sealing material is uncoupled with respect to said contact surface at least prior to affixing said contact surface to said first tissue portion; and a second tissue cover covering at least said layer. The device may be provided in the form of a kit. A system and method including the device are also provided.

In the method claims that follow, alphanumeric characters and Roman numerals used to designate claim steps are provided for convenience only and do not imply any particular order of performing the steps.

It should be noted that the word “comprising” as used throughout the appended claims is to be interpreted to mean “including but not limited to”.

While there has been shown and disclosed example embodiments in accordance with the invention, it will be appreciated that many changes may be made therein without departing from the spirit of the invention.

1-56. (canceled)

57. A device for applying a vacuum to a tissue, comprising:
(A) a first tissue cover defining an internal volume at least in cooperation with the tissue in operation of said device, said first tissue cover comprising first peripheral portion having a tissue facing contact surface adapted to be sealingly affixed to a first tissue portion circumscribing the tissue, said device adapted for enabling a vacuum to be selectively applied to said internal volume in operation of said device;
(B) a substantially contiguous layer of flowable sealing material configured for covering in use at least a second tissue portion abuttingly adjacent at least a part of said first peripheral portion, said sealing material configured for filling gaps between said contact surface and said first tissue portion that may form leaks by flowing towards said contact surface under suction provided when said vacuum is applied to said internal volume, wherein said sealing material is uncoupled with respect to said contact surface at least prior to affixing said contact surface to said first tissue portion; and
(C) a second tissue cover covering at least a portion of said layer.

58. The device according to claim 57, wherein said substantially contiguous layer is abuttingly adjacent the full periphery of said first peripheral portion.

59. The device according to claim 57, wherein said contiguous layer further covers an external portion of said first tissue cover adjacent said first peripheral portion.

60. The device according to claim 57, further comprising an interface comprising at least one member selected from the group consisting of a medication, a lotion, gauze, bandages, and packing material, in said internal volume at least in operation of said device.

61. The device according to claim 57, wherein said first tissue cover comprises a first drape having a tissue-facing adhesive layer and a removable protective layer carried against said adhesive layer prior to use, and wherein said first peripheral portion is adapted to be affixed to the first tissue portion via said adhesive layer having first removed said protective layer, and wherein optionally said protective layer comprises any one of a paper strip, tape and a film.

62. The device according to claim 57, wherein said second tissue cover is configured for retaining said sealant material between said second tissue cover and said first tissue cover and for enabling said sealant material to flow to fill gaps as required during operation of said device.

63. Device according to claim 57, wherein said second tissue cover comprises a second peripheral portion configured for affixing said second peripheral portion to a third tissue portion circumscribing and adjacent said second tissue portion.

64. The device according to claim 57, wherein said sealing material comprises any one of petroleum jelly and vacuum grease.

65. The device according to claim 57, wherein said device is disposable.

66. Device according to claim 57, further comprising a venting arrangement for enabling venting of said internal volume to ambient when in use, wherein optionally said venting arrangement comprises at least one bleeding orifice arrangement having an effective flow area compatible with enabling a desired flow rate to be provided during operation of the device, or wherein optionally said venting arrangement is controllable and adapted for regulating, at least during operation of said system, the vacuum level at said internal volume, and wherein optionally said controllable venting arrangement comprises any one of a pressure regulator and a solenoid valve.

67. Device according to claim 57, further comprising a port adapted for connection to an irrigation source for providing, at least during operation of said device, fluid communication between said internal volume and said irrigation source, such as to enable a desired irrigation material to be provided to the internal volume.

68. The device according to claim 57, wherein said first tissue cover comprises a conduit having at one end thereof an inlet port opening into said internal volume and an outlet port at another end thereof adapted for connection to a suitable vacuum pump.

69. The device according to claim 57, further comprising a pump head having a pumping chamber including an inlet and an outlet, wherein said pump head is integrated with said first tissue cover, wherein said inlet is comprised in said first tissue cover and is in fluid communication therewith, and wherein said outlet is connectable to a powered drive apparatus comprising a drive unit coupled to a primary pump, in operation said powered drive apparatus being configured for operating said pump head via a non-mechanical coupling arrangement when driven by said drive unit and adapted for selectively providing a predetermined working pressure within said internal volume below ambient pressure of an external environment via operation of said pump head.

70. The device according to claim 57, wherein said device is in the form of a kit, comprising:
(A) said first tissue cover;
(B) said flowable sealing material; and
(C) said second tissue cover.

71. A system for applying a vacuum to a tissue, comprising:
a device comprising
(A) a first tissue cover defining an internal volume at least in cooperation with the tissue in operation of said device, said first tissue cover comprising first peripheral portion having a tissue facing contact surface adapted to be sealingly affixed to a first tissue portion circumscribing the tissue, said device adapted for enabling a vacuum to be selectively applied to said internal volume in operation of said device;
(B) a substantially contiguous layer of flowable sealing material configured for covering in use at least a second tissue portion abuttingly adjacent at least a part of said first peripheral portion, said sealing material configured for filling gaps between said contact surface and said first tissue portion that may form leaks by flowing towards said contact surface under suction provided when said vacuum is applied to said internal volume, wherein said sealing material is uncoupled with respect to said contact surface at least prior to affixing said contact surface to said first tissue portion; and
(C) a second tissue cover covering at least a portion of said layer; and
a vacuum source configured for generating a vacuum and applying said vacuum to said internal volume.

72. A method for providing a self sealing enclosure for use over a target body part to facilitate application of a vacuum to the body part, comprising:
(a) providing an enclosure comprising a device including a first tissue cover defining an internal volume at least in cooperation with the body part in operation of said device, said first tissue cover comprising a first peripheral portion adapted to be sealingly affixed to a first tissue portion circumscribing the targeted body part, said device adapted for enabling a vacuum to be selectively applied to said internal volume in operation of said device;
(b) placing said first tissue cover over a the body part,
(d) sealingly affixing said first peripheral portion to the first tissue portion;
(e) subsequently applying a flowable sealing material to cover in a substantially contiguous layer at least a second tissue portion abuttingly adjacent at least a part of said first peripheral portion, said sealing material configured for filling gaps between said first peripheral portion and said first tissue portion that may form leaks when a vacuum is applied to said internal volume; and
(f) covering at least a portion of said layer with a second tissue cover.

73. The method according to claim 72, wherein in step (e) said substantially contiguous layer is abuttingly adjacent the full periphery of said first peripheral portion, and wherein in step (d) said sealing material is further applied to an outside surface of said first tissue cover adjacent said first peripheral portion such that said contiguous layer extends to said outside of said first tissue cover.

74. The method according to claim 73, wherein said sealing material is applied to one of (i) substantially the full said outside surface, wherein further to seal said outside surface, and (ii) a third peripheral portion of said outside surface adjacent said first peripheral portion.

75. The method according to claim 72, further comprising providing, prior to step (b), at least one member selected from the group consisting of a medication, a lotion, gauze, bandages, and packing material, to the tissue.

76. The method according to claim 72, wherein said first tissue cover comprises a first non-rigid drape having a tissue-facing adhesive layer and a protective layer carried against said adhesive layer prior to use, wherein prior to step (c) said protective layer is removed, and in step (c) said first peripheral portion is affixed to the first tissue portion via said adhesive layer.

77. The method according to claim 72, wherein said second tissue cover comprises a second peripheral portion and step (e) further comprises sealingly affixing said second peripheral portion to a third tissue portion circumscribing and adjacent said second tissue portion.

78. The method according to claim 72, wherein in step (d) said second tissue portion circumscribes said first peripheral portion.

79. The method according to claim 72, wherein said sealing material comprises any one of petroleum jelly and vacuum grease.
